(19) United States
(12) Patent Application Publication
(10) Pub. No.: US 2004/0242662 A1
(43) Pub. Date: Dec. 2, 2004

(54) Azinyl sulfonylimidazoles for use as microbicidal agents

(76) Inventors: Stefan Hillebrand, Neuss (DE); Lutz Assmann, Langenfeld (DE); Ulrike Wachendorff-Neumann, Neuwied (DE)

Correspondence Address: Bayer CropScience, Patent Department, 100 Bayer Road, Pittsburgh, PA, 15205-9741, US

(21) Appl. No.: 10/451,208

(22) PCT Filed: Dec. 5, 2001
(86) PCT No.: PCT/EP01/14241

(30) Foreign Application Priority Data
Dec. 18, 2000 (DE) 100631169

(51) Int. Cl. A61K 31/4164; C07D 233/52

(52) U.S. Cl. 514/398; 548/331.1

(57) ABSTRACT

Novel azinylsulfonylimidazoles of the formula

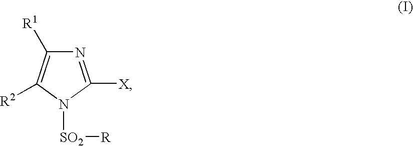

in which

R¹ represents hydrogen, halogen, cyano, nitro, alkyl, halogenoalkyl, optionally substituted aryl or optionally substituted aralkyl,

R² represents hydrogen, halogen, cyano, nitro, alkyl, halogenoalkyl, optionally substituted aryl or optionally substituted aralkyl,

R¹ and R² together with the carbon atoms to which they are attached represent an optionally substituted ring,

R is as defined in the description,

and

X represents halogen, alkyl, halogenoalkyl, cyano or thiocarbamoyl,

a process for preparing the novel compounds and their use for controlling unwanted microorganisms.

The present invention relates to novel azinylsulfonylimidazoles, to a process for their preparation and to their use for controlling unwanted microorganisms.

It is already known that certain sulfonylimidazoles have microbicidal and in particular fungicidal properties (cf. WO 97-06 171, WO 97-47 620, WO 98-39 331 and WO 99-05 116). The activity of these compounds is good; however, at low application rates it is sometimes unsatisfactory.

This invention now provides novel azinylsulfonylimidazoles of the formula

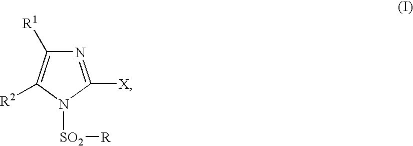

in which

R¹ represents hydrogen, halogen, cyano, nitro, alkyl, halogenoalkyl, optionally substituted aryl or optionally substituted aralkyl,

R² represents hydrogen, halogen, cyano, nitro, alkyl, halogenoalkyl, optionally substituted aryl or optionally substituted aralkyl,

R¹ and R² together with the carbon atoms to which they are attached represent an optionally substituted ring,

R represents a radical of the formula

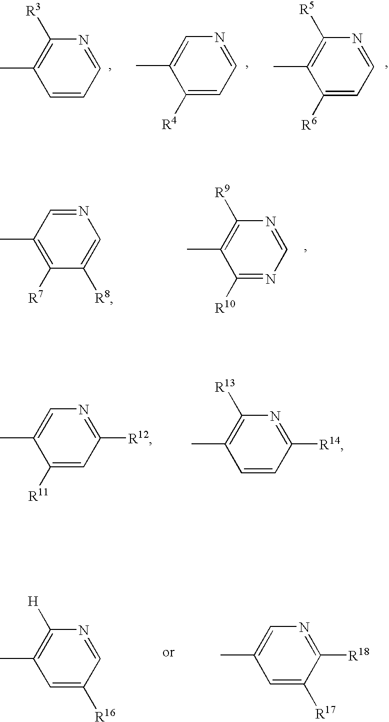

in which

R³, R⁴, R⁵, R⁶, R⁷, R⁸, R⁹, R¹⁰, R¹¹, R¹², R¹³, R¹⁴, R¹⁵, R¹⁶, R¹⁷ and R¹⁸ independently of one another represent fluorine, chlorine, bromine or methoxy,

or

R represents a radical of the formula

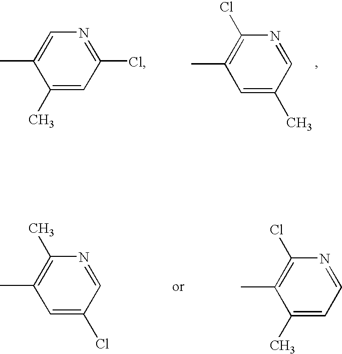

and

X represents halogen, alkyl, halogenoalkyl, cyano or thiocarbamoyl.

Furthermore, it has been found that azinylsulfonylimidazoles of the formula (I) can be prepared by reacting imidazoles of the formula

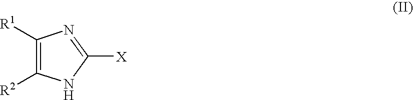

in which

R¹, R² and X are as defined above,

with sulfonyl halides of the formula

R—SO₂—X¹ (III),

in which

R is as defined above and

X¹ represents chlorine or bromide,

if appropriate in the presence of an acid binder and if appropriate in the presence of a diluent.

Finally, it has been found that the azinylsulfonylimidazoles of the formula (I) have very good microbicidal properties and can be used both in crop protection and in the protection of materials for controlling unwanted microorganisms.

Surprisingly, the azinylsulfonylimidazoles of the formula (I) according to the invention have considerably better fungicidal activity than the constitutionally most similar prior-art compounds of the same direction of action.

The formula (I) provides a general definition of the azinylsulfonylimidazoles according to the invention. Preference is given to those compounds of the formula (I) in which

R¹ represents hydrogen, fluorine, chlorine, bromine, iodine, cyano, nitro, alkyl having 1 to 4 carbon atoms or halogenoalkyl having 1 to 4 carbon atoms and 1 to 9 halogen atoms, or represents aryl having 6 to 10 carbon atoms, where these aryl radicals may be mono- to trisubstituted by identical or different substituents from the group consisting of fluorine, chlorine, bromine, iodine, cyano, nitro, alkyl having 1 to 4 carbon atoms, alkoxy having 1 to 4 carbon atoms and halogenoalkyl having 1 to 4 carbon atoms and 1 to 9 identical or different halogen atoms, or

represents aralkyl having 6 to 10 carbon atoms in the aryl moiety and 1 to 4 carbon atoms in the alkyl moiety, where these radicals may be mono- to trisubstituted in the aryl moiety by identical or different substituents from the group consisting of fluorine, chlorine, bromine, iodine, cyano, nitro, alkyl having 1 to 4 carbon atoms, alkoxy having 1 to 4 carbon atoms and/or halogenoalkyl having 1 to 4 carbon atoms and 1 to 9 identical or different halogen atoms,

R² represents hydrogen, fluorine, chlorine, bromine, iodine, cyano, nitro, alkyl having 1 to 4 carbon atoms, or halogenoalkyl having 1 to 4 carbon atoms and 1 to 9 halogen atoms, or represents aryl having 6 to 10 carbon atoms, where the aryl radical may be mono- to trisubstituted by identical or different substituents from the group consisting of fluorine, chlorine, bromine, iodine, cyano, nitro, alkyl having 1 to 4 carbon atoms, alkoxy having 1 to 4 carbon atoms and halogenoalkyl having 1 to 4 carbon atoms and 1 to 9 identical or different halogen atoms, or

represents aralkyl having 6 to 10 carbon atoms in the aryl moiety and 1 to 4 carbon atoms in the alkyl moiety, where these radicals may be mono- to trisubstituted in the aryl moiety by identical or different substituents from the group consisting of fluorine, chlorine, bromine, iodine, cyano, nitro, alkyl having 1 to 4 carbon atoms, alkoxy having 1 to 4 carbon atoms and halogenoalkyl having 1 to 4 carbon atoms and 1 to 9 identical or different halogen atoms, or

R¹ and R² together with the carbon atoms to which they are attached represent a five- or six-membered ring which may be mono- to trisubstituted by identical or different substituents from the group consisting of halogen and alkyl having 1 to 4 carbon atoms, or

R¹ and R² together with the carbon atoms to which they are attached represent a bicyclic ring system having 5 or 6 members per ring, where the bicyclic ring system may be mono- to tetrasubstituted by identical or different substituents from the group consisting of halogen and alkyl having 1 to 4 carbon atoms,

R represents a radical of the formula

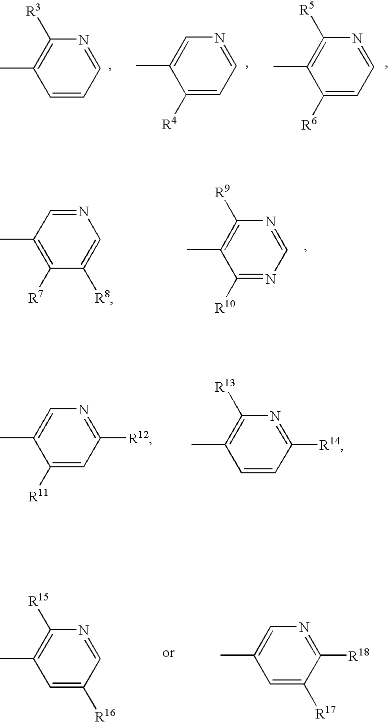

in which

R³, R⁴, R⁵, R⁶, R⁷, R⁸, R⁹, R¹⁰, R¹¹, R¹², R¹³, R¹⁴, R¹⁵, R¹⁶, R¹⁷ and R¹⁸ independently of one another represent fluorine, chlorine, bromine or methoxy,

or

R represents a radical of the formula

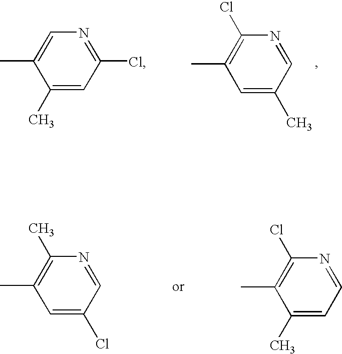

and

X represents fluorine, chlorine, bromine, alkyl having 1 to 4 carbon atoms, halogenoalkyl having 1 to 4 carbon atoms and 1 to 9 halogen atoms, cyano or thiocarbamoyl.

Particular preference is given to azinylsulfonylimidazoles of the formula (I), in which

R¹ represents hydrogen, fluorine, chlorine, bromine, cyano, nitro, methyl, trifluormethyl, ethyl, phenyl, tolyl, methoxyphenyl, chlorophenyl, trifluoromethylphenyl, benzyl, methylbenzyl, methoxybenzyl, chlorbenzyl or trifluoromethylbenzyl,

R² represents hydrogen, fluorine, chlorine, bromine, cyano, nitro, methyl, trifluoromethyl, ethyl, phenyl, tolyl, methoxyphenyl, chlorophenyl, trifluoromethylphenyl, benzyl, methylbenzyl, methoxybenzyl, chlorobenzyl or trifluoromethylbenzyl, or

R¹ and R² together with the carbon atoms to which they are attached represent phenylene which may be mono- to trisubstituted by identical or different substituents from the group consisting of fluorine, chlorine, bromine, methyl, ethyl and trifluoromethyl, or

R¹ and R² together with the carbon atoms to which they are attached represent methylenedioxyphenylene which may be mono- or disubstituted at the methylene group by identical or different substituents from the group consisting of fluorine, chlorine, bromine, methyl and trifluoromethyl, or

R¹ and R² together with the carbon atoms to which they are attached represent ethylenedioxyphenylene which may be mono- to tetrasubstituted by identical or different substituents from the group consisting of fluorine, chlorine, bromine, methyl and trifluoromethyl,

R represents a radical of the formula

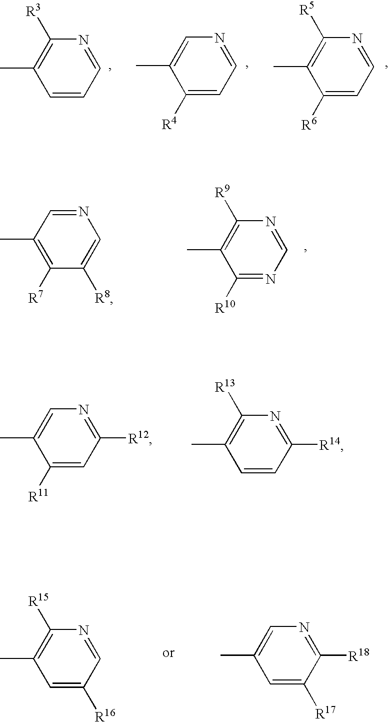

in which

R³, R⁴, R⁵, R⁶, R⁷, R⁸, R⁹, R¹⁰, R¹¹, R¹², R¹³, R¹⁴, R¹⁵, R¹⁶, R¹⁷ and R¹⁸ independently of one another represent fluorine, chlorine, bromine or methoxy,

or

R represents a radical of the formula

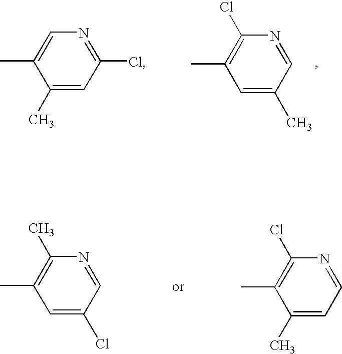

and

X represents fluorine, chlorine, bromine, methyl, trifluoromethyl, cyano or thiocarbamoyl.

Using 2,5-bibromo4-methyl-1H-imidazole and 2-chloro-3-pyridinesulfonyl chloride as starting materials, the course of the process according to the invention can be illustrated by the formula below.

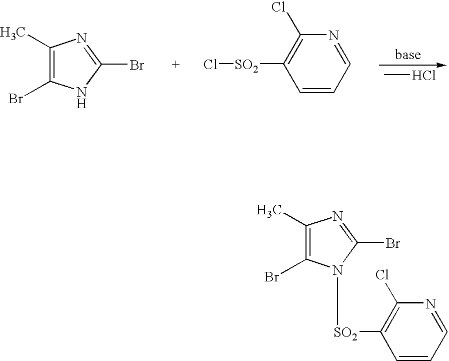

The formula (II) provides a general definition of the imidazole required as starting materials for carrying out the process according to the invention. In this formula, R¹, R² and X preferably have those meanings which have already been mentioned in connection with the description of the compounds of the formula (I) according to the invention as being preferred for theses substituents. These imidazoles can be present in the two tautomeric forms below.

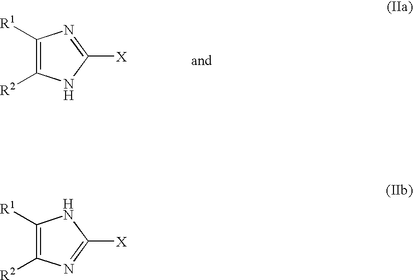

If the molecular unit

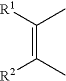

is symmetric, the tautomeric forms are identical. Accordingly, in this case the reaction according to the invention with sulfonyl halide of the formula (III) gives in each case only one particular end product of the formula (I), independently of whichever of the two tautomeric forms reacts.

If the molecular unit

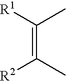

is asymmetric, the two tautomeric forms are not identical. Accordingly, in this case the reaction according to the invention with sulfonyl halide of the formula (III) may give end products of the formula (I) which are derived from the tautomeric forms (IIa) and/or (IIb). If both tautomers are reacting, this gives end products of the formula (I) in the form of mixtures.

The imidazoles of the formula (II) are known or can be prepared by known methods (cf. WO 97-06171, Chem. Ber. 85, (1952) 1012-1020, J. Med. Chem. 34 (1991), 1110-1116 and J. Chem. Soc. 1965, 3017-3021).

The formula (III) provides a general definition of the sulfonyl halides further required as starting materials for carrying out the process according to the invention. In this formula, R preferably has those meanings which have already been mentioned in connection with the description of the compounds of the formula (I) according to the invention as being preferred for this radical. X¹ preferably represents chlorine.

The sulfonyl halides of formula (III) are known or can be prepared by known methods (cf. DE-A 198 40 319, U.S. Pat. No. 5,858,924 and EP-A 0 457 581).

Suitable diluents for carrying out the process according to the invention are all customary organic solvents which are poorly miscible with water, Preference is given to using aliphatic, alicyclic or aromatic hydrocarbons, such as petroleum ether, hexane, heptane, cyclohexane, methylcyclohexane, benzene, toluene, xylene or decaline; furthermore halogenated hydrocarbons, such as chlorobenzene, dichlorobenzene, dichloromethane, chloroform, carbon tetrachloride, dichloroethane or trichloroethane; furthermore ethers, such as diethyl ether, diisopropyl ether, methyl t-butyl ether, methyl t-amyl ether, dioxane, tetrahydrofuran, 1,2-dimethoxyethane, 1,2-diethoxyethane or anisole; and esters, such as methyl acetate or ethyl acetate.

Preferred acid binders for carrying out the process according to the invention are alkaline earth metal or alkali metal hydroxides, acetates, carbonates or bicarbonates. Examples which may be mentioned are sodium hydroxide, potassium hydroxide, sodium acetate, potassium acetate, calcium acetate, sodium carbonate, potassium carbonate, potassium bicarbonate. It is furthermore possible to use ammonium compounds and organic bases, such as ammonium acetate or ammonium carbonate, or tertiary amines, such as trimethylamine, triethylamine, tributylamine, N,N-dimethylaniline, N,N-dimethylbenzylamine, pyridine, N-methylpiperidine, N-methylmorpholine, N,N-dimethylaminopyridine, diazabicyclooctane (DABCO), diazabicyclononene (DBN) or diazabicycloundecene (DBU).

When carrying out the process according to the invention, the reaction temperatures can be varied within a relatively wide range. In general, the process is carried out at temperatures between 0° C. and 130° C., preferably between 20° C. and 50° C.

The process according to the invention is generally carried out under atmospheric pressure. However, it is also possible to operate under elevated or reduced pressure, for example, under pressures between 0.1 bar and 10 bar

When carrying out the process according to the invention, in general an equivalent amount or else an excess of sulfonyl halide of the formula (III) and an equivalent amount or else an excess of acid binder are employed per mole of imidazole of the formula (II). Work-up is carried out by customary methods.

The compounds according to the invention have potent microbicidal activity and can be employed for controlling undesirable microorganisms, such as fungi and bacteria, in crop protection and in the protection of materials.

Fungicides can be employed in crop protection for controlling Plasmodiophoromycetes, Oomycetes, Chytridiomycetes, Zygomycetes, Ascomycetes, Basidiomycetes and Deuteromycetes.

Bactericides can be employed in crop protection for controlling Pseudomonadaceae, Rhizobiaceae, Enterobacteriaceae, Corynebacteriaceae and Streptomycetaceae.

Some pathogens causing fungal and bacterial diseases which come under the generic names listed above may be mentioned as examples, but not by way of limitation:

Xanthomonas species, such as, for example, Xanthomonas campestris pv. oryzae;

Pseudomonas species, such as, for example, Pseudomonas syringae pv. lachrymans;

Erwinia species, such as, for example, Erwinia amylovora;

Pythium species, such as, for example, Pythium ultimum;

Phytophthora species, such as, for example, Phytophthora infestans;

Pseudoperonospora species, such as, for example, Pseudoperonospora humuli or Pseudoperonospora cubensis;

Plasmopara species, such as, for example, Plasmopara viticola;

Bremia species, such as, for example, Bremia lactucae;

Peronospora species, such as, for example, Peronospora pisi or P. brassicae;

Erysiphe species, such as, for example, Erysiphe graminis;

Sphaerotheca species, such as, for example, Sphaerotheca fuliginea;

Podosphaera species, such as, for example, Podosphaera leucotricha;

Venturia species, such as, for example, Venturia inaequalis;

Pyrenophora species, such as, for example, Pyrenophora teres or P. graminea (conidia form: Drechslera, syn: Helminthosporium);

Cochliobolus species, such as, for example, Cochliobolus sativus (conidia form: Drechslera, syn: Helminthosporium);

Uromyces species, such as, for example, Uromyces appendiculatus;

Puccinia species, such as, for example, Puccinia recondita;

Sclerotinia species, such as, for example, Sclerotinia sclerotiorum;

Tilletia species, such as, for example, Tilletia caries;

Ustilago species, such as, for example, Ustilago nuda or Ustilago avenae;

Pellicularia species, such as, for example, Pellicularia sasakii;

Pyricularia species, such as, for example, Pyricularia oryzae;

Fusarium species, such as, for example, Fusarium culmorum;

Botrytis species, such as, for example, Botrytis cinerea;

Septoria species, such as, for example, Septoria nodorum;

Leptosphaeria species, such as, for example, Leptosphaeria nodorum;

Cercospora species, such as, for example, Cercospora canescens;

Alternaria species, such as, for example, Alternaria brassicae; and

Pseudocercosporella species, such as, for example, Pseudocercosporella herpotrichoides.

The fact that the active compounds are well tolerated by plants at the concentrations required for controlling plant diseases permits the treatment of above-ground parts of plants, of propagation stock and seeds, and of the soil.

The active compounds according to the invention can be used with particularly good results for controlling diseases in viticulture and in fruit and vegetable growing, such as, for example, against Phytophtora species.

The active compounds according to the invention are also suitable for increasing the, yield of crops. In addition, they show reduced toxicity and are well tolerated by plants.

If appropriate, the active compounds according to the invention can, at certain concentrations and application rates, also be employed as herbicides, for regulating plant growth and for controlling animal pests. If appropriate, they can also be used as intermediates or precursors in the synthesis of other active compounds.

According to the invention, it is possible to treat all plants and parts of plants. Plants are to be understood here as meaning all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including plant cultivars which can or cannot be protected by plant breeders' certificates. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested material and vegetative and generative propagation material, for example seedlings, tubers, rhizomes, cuttings and seeds.

The treatment of the plants and parts of plants according to the invention with the active compounds is carried out directly or by action on their environment, habitat or storage area according to customary treatment methods, for example by dipping, spraying, evaporating, atomizing, broadcasting, brushing-on, injecting and, in the case of propagation material, in particular in the case of seeds, furthermore by one- or multilayer coating.

In the protection of materials, the compounds according to the invention can be employed for protecting industrial materials against infection with, and destruction by, undesired microorganisms.

Industrial materials in the present context are understood as meaning non-living materials which have been prepared for use in industry. For example, industrial materials which are intended to be protected by active compounds according to the invention from microbial change or destruction can be tackifiers, sizes, paper and board, textiles, leather, wood, paints and plastic articles, cooling lubricants and other materials which can be infected with, or destroyed by, microorganisms. Parts of production plants, for example cooling-water circuits, which may be impaired by the proliferation of microorganisms may also be mentioned within the scope of the materials to be protected. Industrial materials which may be mentioned within the scope of the present invention are preferably tackifiers, sizes, paper and board, leather, wood, paints, cooling lubricants and heat-transfer liquids, particularly preferably wood.

Microorganisms capable of degrading or changing the industrial materials which may be mentioned are, for example, bacteria, fungi, yeasts, algae and slime organisms. The active compounds according to the invention preferably act against fungi, in particular molds, wood-discoloring and wood-destroying fungi (Basidiomycetes) and against slime organisms and algae.

Microorganisms of the following genera may be mentioned as examples:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Sclerophoma, such as Sclerophoma pityophila,

Trichoderma, such as Trichoderma viride,

Escherichia, such as Escherichia coli,

Pseudomonas, such as Pseudomonas aeruginosa, and

Staphylococcus, such as Staphylococcus aureus.

Depending on their particular physical and/or chemical properties, the active compounds can be converted into the customary formulations, such as solutions, emulsions, suspensions, powders, foams, pastes, granules, aerosols and microencapsulations in polymeric substances and in coating compositions for seeds, and ULV cool and warm fogging formulations.

These formulations are produced in a known manner, for example by mixing the active compounds with extenders, that is liquid solvents, liquefied gases under pressure, and/or solid carriers, optionally with the use of surfactants, that is emulsifiers and/or dispersants, and/or foam formers. If the extender used is water, it is also possible to employ, for example, organic solvents as auxiliary solvents. Essentially, suitable liquid solvents are: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, alcohols such as butanol or glycol and their ethers and esters, ketones such as acetone, methyl ethyl ketone, methyl isobutyl ketone or cyclohexanone, strongly polar solvents such as dimethylformamide or dimethyl sulfoxide, or else water. Liquefied gaseous extenders or carriers are to be understood as meaning liquids which are gaseous at standard temperature and under atmospheric pressure, for example aerosol propellants such as halogenated hydrocarbons, or else butane, propane, nitrogen and carbon dioxide. Suitable solid carriers are: for example ground natural minerals such as kaolins, clays, talc, chalk, quartz, attapulgite, montmorillonite or diatomaceous earth, and ground synthetic minerals such as finely divided silica, alumina and silicates. Suitable solid carriers for granules are: for example crushed and fractionated natural rocks such as calcite, marble, pumice, sepiolite and dolomite, or else synthetic granules of inorganic and organic meals, and granules of organic material such as sawdust, coconut shells, maize cobs and tobacco stalks. Suitable emulsifiers and/or foam formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates, or else protein hydrolysates. Suitable dispersants are: for example lignosulfite waste liquors and methylcellulose.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Other possible additives are mineral and vegetable oils.

It is possible to use colorants such as inorganic pigments, for example iron oxide, titanium oxide and Prussian Blue, and organic dyestuffs such as alizarin dyestuffs, azo dyestuffs and metal phthalocyanine dyestuffs, and trace nutrients such as salts of iron, manganese, boron, copper, cobalt, molybdenum and zinc.

The formulations generally comprise between 0.1 and 95 percent by weight of active compound, preferably between 0.5 and 90%.

The active compounds according to the invention can, as such or in their formulations, also be used in a mixture with known fungicides, bactericides, acaricides, nematicides or insecticides, to broaden, for example, the activity spectrum or to prevent development of resistance. In many cases, synergistic effects are obtained, i.e. the activity of the mixture is greater than the activity of the individual components.

Suitable mixing components are, for example, the following compounds:

Fungicides:

aldimorph, ampropylfos, ampropylfos-potassium, andoprim, anilazine, azaconazole, azoxystrobin,

benalaxyl, benodanil, benomyl, benzamacril, benzamacryl-isobutyl, bialafos, binapacryl, biphenyl, bitertanol, blasticidin-S, bromuconazole, bupirimate, buthiobate,

calcium polysulfide, capsimycin, captafol, captan, carbendazim, carboxin, carvone, quinomethionate, chlobenthiazone, chlorfenazole, chloroneb, chloropicrin, chlorothalonil, chlozolinate, clozylacon, cufraneb, cymoxanil, cyproconazole, cyprodinil, cyprofuram, carpropamid,

debacarb, dichlorophen, diclobutrazole, diclofluanid, diclomezine, dicloran, diethofencarb, difenoconazole, dimethirimol, dimethomorph, diniconazole, diniconazole-M, dinocap, diphenylamine, dipyrithione, ditalimfos, dithianon, dodemorph, dodine, drazoxolon,

edifenphos, epoxiconazole, etaconazole, ethirimol, etridiazole,

famoxadone, fenapanil, fenarimol, fenbuconazole, fenfuram, fenitropan, fenpiclonil, fenpropidin, fenpropimorph, fentin-acetate, fentin-hydroxide, ferbam, ferimzon, fluazinam, flumetover, fluoromide, fluquinconazole, flurprimidol, flusilazole,

flusulfamiide, flutolanil, flutriafol, folpet, fosetyl-aluminum, fosetyl-sodium, fthalide, fuberidazole, furalaxyl, furametpyr, furcarbonil, furconazole, furconazole-cis, furmecyclox, fenhexamid,

guazatine,

hexachlorobenzene, hexaconazole, hymexazole,

imazalil, imnibenconazole, iminoctadin, iminoctadine albesilate, iminoctadine triacetate, iodocarb, ipconazole, iprobenfos (IBP), iprodione, irumamycin, isoprothiolane, isovaledione, iprovalicarb,

kasugamycin, kresoxim-methyl, copper preparations such as copper hydroxide, copper naphthenate, copper oxychloride, copper sulfate, copper oxide, oxine copper and Bordeaux mixture,

mancopper, mancozeb, maneb, meferimzone, mepanipyrim, mepronil, metalaxyl, metconazole, methasulfocarb, methfuroxam, metiram, metomeclam, metsulfovax, mildiomycin, myclobutanil, myclozolin,

nickel dimethyldithiocarbamate, nitrothal-isopropyl, nuarimol,

ofurace, oxadixyl, oxamocarb, oxolinic acid, oxycarboxim, oxyfenthiin,

paclobutrazole, pefurazoate, penconazole, pencycuron, phosdiphen, pimaricin, piperalin, polyoxin, polyoxorim, probenazole, prochloraz, procymidone, propamocarb, propanosine-sodium, propiconazole, propineb, pyrazophos, pyrifenox, pyrimethanil, pyroquilon, pyroxyfur,

quinconazole, quintozen (PCNB), quinoxyfen,

sulfur and sulfur preparations, spiroxamine,

tebuconazole, tecloftalam, tecnazene, tetcyclacis, tetraconazole, thiabendazole, thicyofen, thifluzamide, thiophanate-methyl, thiram, tioxymid, tolclofos-methyl, tolylfluanid, triadimefon, triadimenol, triazbutil, triazoxide, trichlamid, tricyclazole, tridemorph, triflumizole, triforine, triticonazole, trifloxystrobin,

uniconazole,

validamycin A, vinclozolin, vinicornazole,

zarilarid, zineb, ziram and

Dagger G,

OK-8705,

OK-8801,

α-(1,1-dimethylethyl)-β-(2-phenoxyethyl)-1H-1,2,4-triazole-1-ethanol,

α-(2,4-dichlorophenyl)-β-fluoro-b-propyl-1H-1,2,4-triazole-1-ethanol,

α-(2,4-dichlorophenyl)-β-methoxy-a-methyl-1H-1,2,4-triazole-1-ethanol,

α-(5-methyl-1,3-dioxan-5-yl)-β-[[4-(trifluoromethyl)-phenyl]-methylene]-1H-1,2,4-triazole-1-ethanol,

(5RS,6RS)-6-hydroxy-2,2,7,7-tetramethyl-5-(1H-1,2,4-triazol-1-yl)-3-octanone,

(E)-a-(methoxyimino)-N-methyl-2-phenoxy-phenylacetamide,

1-(2,4-dichlorophenyl)-2-(1H-1,2,4-triazol-1-yl)-ethanone O-(phenylmethyl)-oxime,

1-(2-methyl-1-naphthalenyl)-1H-pyrrol-2,5-dione,

1-(3,5-dichlorophenyl)-3-(2-propenyl)-2,5-pyrrolidindione,

1-[(diiodomethyl)-sulfonyl]-4-methyl-benzene,

1-[[2-(2,4-dichlorophenyl)-1,3-dioxolan-2-yl]-methyl]-1H-imidazole,

1-[[2-(4-chlorophenyl)-3-phenyloxiranyl]-methyl]-1H-1,2,4-triazole,

1-[1-[2-[(2,4-dichlorophenyl)-methoxy]-phenyl]-ethenyl]-1H-imidazole,

1-methyl-5-nonyl-2-(phenylmethyl)-3-pyrrolidinol,

2′,6′-dibromo-2-methyl-4′-trifluoromethoxy-4′-trifluoro-methyl-1,3-thiazole-5-carboxanilide,

2,6-dichloro-5-(methylthio)4-pyrimidinyl-thiocyanate,

2,6-dichloro-N-(4-trifluoromethylbenzyl)-benzamide,

2,6-dichloro-N-[[4-(trifluoromethyl)-phenyl]-methyl]-benzamide,

2-(2,3,3-triiodo-2-propenyl)-2H-tetrazole,

2-[(1-methylethyl)-sulfonyl]-5-(trichloromethyl)-1,3,4-thiadiazole,

2-[[6-deoxy4-O-(4-O-methyl-β-D-glycopyranosyl)-a-D-glucopyranosyl]-amino]-4-methoxy-1H-pyrrolo[2,3-d]pyrimidine-5-carbonitrile,

2-aminobutane,

2-bromo-2-(bromomethyl)-pentanedinitrile,

2-chloro-N-(2,3-dihydro-1,1,3-trimethyl-1H-inden4-yl)-3-pyridinecarboxamide,

2-chloro-N-(2,6-dimethylphenyl)-N-(isothiocyanatomethyl)-acetamide,

2-phenylphenol (OPP),

3,4-dichloro-1-[4-(difluoromethoxy)-phenyl]-1H-pyrrol-2,5-dione,

3,5-dichloro-N-[cyano[(1-methyl-2-propynyl)-oxy]-methyl]-benzamide,

3-(1,1-dimethylpropyl-1-oxo-1H-indene-2-carbonitrile,

3-[2-(4-chlorophenyl)-5-ethoxy-3-isoxazolidinyl]-pyridine,

4-chloro-2-cyano-N,N-dimethyl-5-(4-methylphenyl)-1H-imidazole-1-sulfonamide,

4-methyl-tetrazolo[1,5-a]quinazolin-5(4H)-one,

8-hydroxyquinoline sulfate,

9H-xanthene-2-[(phenylamino)-carbonyl]-9-carboxylic hydrazide,

bis-(1-methylethyl)-3-methyl4-[(3-methylbenzoyl)-oxy]-2,5-thiophenedicarboxylate,

cis-1-(4-chlorophenyl)-2-(1H-1,2,4-triazol-1-yl)-cycloheptanol,

cis-4-[3-[4-(1,1-dimethylpropyl)-phenyl-2-methylpropyl]-2,6-dimethyl-morpholine hydrochloride,

ethyl [(4-chlorophenyl)-azo]-cyanoacetate,

potassium bicarbonate,

methanetetrathiol-sodium salt,

methyl 1-(2,3-dihydro-2,2-dimethyl-1H-inden-1-yl)-1H-imidazole-5-carboxylate,

methyl N-(2,6-dimethylphenyl)-N-(5-isoxazolylcarbonyl)-DL-alaninate,

methyl N-(chloroacetyl)-N-(2,6-dimethylphenyl)-DL-alaninate,

N-(2,6-dimethylphenyl)-2-methoxy-N-(tetrahydro-2-oxo-3-furanyl)-acetamide,

N-(2,6-dimethylphenyl)-2-methoxy-N-(tetrahydro-2-oxo-3-thienyl)-acetamide,

N-(2-chloro4-nitrophenyl)-4-methyl-3-nitro-benzenesulfonamide,

N-(4-cyclohexylphenyl)-1,4,5,6-tetrahydro-2-pyrimidinamine,

N-(4-hexylphenyl)-1,4,5,6-tetrahydro-2-pyrimidinamine,

N-(5-chloro-2-methylphenyl)-2-methoxy-N-(2-oxo-3-oxazolidinyl)-acetamide,

N-(6-methoxy)-3-pyridinyl-cyclopropanecarboxamide,

N-[2,2,2-trichloro-1-[(chloroacetyl)-amino]-ethyl]-benzamide,

N-[3-chloro4,5-bis(2-propinyloxy)-phenyl]-N′-methoxy-methanimidamide,

N-formyl-N-hydroxy-DL-alanine-sodium salt,

O,O-diethyl [2-(dipropylamino)-2-oxoethyl]-ethylphosphoramidothioate,

O-methyl S-phenyl phenylpropylphosphoramidothioate,

S-methyl 1,2,3-benzothiadiazole-7-carbothioate,

spiro[2H]-1-benzopyrane-2,1′(3′H)-isobenzofuran]-3′-one,

Bactericides:

bronopol, dichlorophen, nitrapyrin, nickel dimethyldithiocarbamate, kasugamycin, octhilinone, furancarboxylic acid, oxytetracyclin, probenazole, streptomycin, tecloftalam, copper sulfate and other copper preparations.

Insecticides/acaricides/nematicides:

abamectin, acephate, acetamiprid, acrinathrin, alanycarb, aldicarb, aldoxycarb, alphacypermethrin, alphamethrin, amitraz, avermectin, AZ 60541, azadirachtin, azamethiphos, azinphos A, azinphos M, azocyclotin,

Bacillus popilliae, Bacillus sphaericus, Bacillus subtilis, Bacillus thuringiensis, baculoviruses, Beauveria bassiana, Beauveria tenella, bendiocarb, benfuracarb, bensultap, benzoximate, beta cyfluthrin, bifenazate, bifenthrin, bioethanomethrin, biopermethrin, BPMC, bromophos A, bufencarb, buprofezin, butathiofos, butocarboxim, butylpyridaben,

cadusafos, carbaryl, carbofuran, carbophenothion, carbosulfan, cartap, chloethocarb, chlorethoxyfos, chlorfenapyr, chlorfenvinphos, chlorfluazuron, chlormephos, chlorpyrifos, chlorpyrifos M, chlovaporthrin, cis-resmethrin, cispermethrin, clocythrin, cloethocarb, clofentezine, cyanophos, cycloprene, cycloprothrin, cyfluthrin, cyhalothrin, cyhexatin, cypermethrin, cyromazine,

deltamethrin, demeton M, demeton S, demeton S-methyl, diafenthiuron, diazinon, dichlorvos, dicofol, diflubenzuron, dimethoate, dimethylvinphos, diofenolan, disulfoton, docusat-sodium, dofenapyn,

eflusilanate, emamectin, empenthrin, endosulfan, Entomopfthora spp., esfenvalerate, ethiofencarb, ethion, ethoprophos, etofenprox, etoxazole, etrimfos,

fenamiphos, fenazaquin, fenbutatin oxide, fenitrothion, fenothiocarb, fenoxacrim, fenoxycarb, fenpropathrin, fenpyrad, fenpyrithrin, fenpyroximate, fenvalerate, fipronil, fluazuron, flubrocythrinate, flucycloxuron, flucythrinate, flufenoxuron, flutenzin, fluvalinate, fonophos, fosmethilan, fosthiazate, fubfenprox, furathiocarb,

granulosis viruses,

halofenozide, HCH, heptenophos, hexaflumuron, hexythiazox, hydroprene,

imidacloprid, isazofos, isofenphos, isoxathion, ivermectin,

nuclear polyhedrosis viruses,

lambda-cyhalothrin, lufenuron,

malathion, mecarbam, metaldehyde, methamidophos, Metharhizium anisopliae, Metharhizium flavoviride, methidathion, methiocarb, methomyl, methoxyfenozide, metolcarb, metoxadiazone, mevinphos, milbemectin, monocrotophos,

naled, nitenpyram, nithiazine, novaluron,

omethoate, oxamyl, oxydemethon-M,

Paecilomyces fumosoroseus, parathion A, parathion M, permethrin, phenthoate, phorate, phosalone, phosmet, phosphamidon, phoxim, pirimicarb, pirimiphos A, pirimiphos M, profenofos, promecarb, propoxur, prothiofos, prothoate, pymetrozine, pyraclofos, pyresmethrin, pyrethrum, pyridaben, pyridathion, pyrimidifen, pyriproxyfen,

quinalphos,

ribavirin,

salithion, sebufos, silafluofen, spinosad, sulfotep, sulprofos,

tau-fluvalinate, tebufenozide, tebufenpyrad, tebupirimiphos, teflubenzuron, tefluthrin, temephos, temivinphos, terbufos, tetrachlorvinphos, theta-cypermethrin, thiamethoxam, thiapronil, thiatriphos, thiocyclam hydrogenoxalate, thiodicarb, thiofanox, thuringiensin, tralocythrin, tralomethrin, triarathene, triazarnate, triazophos, triazuron, trichlophenidine, trichlorfon, triflumuron, trimethacarb,

vamidothion, vaniliprole, Verticillium lecanii,

YI 5302,

zeta-cypermethrin, zolaprofos,

(1R-cis)-[5-(phenylmethyl)-3-furanyl]-methyl 3-[(dihydro-2-oxo-3(2H)-furanylidene)-methyl]-2,2-dimethylcyclopropanecarboxylate,

(3-phenoxyphenyl)-methyl 2,2,3,3-tetramethylcyclopropanecarboxylate,

1-[(2-chloro-5-thiazolyl)methyl]tetrahydro-3,5-dimethyl-N-nitro-1,3,5-triazine-2(1H)-imine,

2-(2-chloro-6-fluorophenyl)4-[4-(1,1-dimethylethyl)phenyl]-4,5-dihydro-oxazole,

2-(acetyloxy)-3-dodecyl-1,4-naphthalenedione,

2-chloro-N-[[[4-(1-phenylethoxy)-phenyl]-amino]-carbonyl]-benzamide,

2-chloro-N-[[[4-(2,2-dichloro-1,1-difluoroethoxy)-phenyl]-amino]-carbonyl]-benzamide,

3-methylphenyl propylcarbamate,

4-[4-(4-ethoxyphenyl)-4-methylpentyl]-1-fluoro-2-phenoxy-benzene,

4-chloro-2-(1,1-dimethylethyl)-5-[[2-(2,6-dimethyl-4-phenoxyphenoxy)ethyl]thio]-3(2H)-pyridazinone,

4-chloro-2-(2-chloro-2-methylpropyl)-5-[(6-iodo-3-pyridinyl)methoxy]-3(2H)-pyridazinone,

4-chloro-5-[(6-chloro-3-pyridinyl)methoxy]-2-(3,4-dichlorophenyl)-3(2H)-pyridazinone,

Bacillus thuringiensis strain EG-2348,

[2-benzoyl-1-(1,1-dimethylethyl)-hydrazinobenzoic acid,

2,2-dimethyl-3-(2,4-dichlorophenyl)-2-oxo-1-oxaspiro[4.5]dec-3-en-4-yl butanoate,

[3-[(6-chloro-3-pyridinyl)methyl]-2-thiazolidinylidene]-cyanamide,

dihydro-2-(nitromethylene)-2H-1,3-thiazine-3(4H)-carboxaldehyde,

ethyl [2-[[1,6-dihydro-6-oxo-1-(phenylmethyl)-4-pyridazinyl]oxy]ethyl]-carbamate,

N-(3,4,4-trifluoro-1-oxo-3-butenyl)-glycine,

N-(4-chlorophenyl)-3-[4-(difluoromethoxy)phenyl]-4,5-dihydro-4-phenyl-1H-pyrazole-1-carboxamide,

N-[(2-chloro-5-thiazolyl)methyl]-N′-methyl-N″-nitro-guanidine,

N-methyl-N′-(1-methyl-2-propenyl)-1,2-hydrazinedicarbothioamide,

N-methyl-N′-2-propenyl-1,2-hydrazinedicarbothioamide,

O,O-diethyl-[2-(dipropylamino)-2-oxoethyl]-ethylphosphoramidothioate,

A mixture with other known active compounds, such as herbicides, or with fertilizers and growth regulators is also possible.

In addition, the compounds of the formula (I) according to the invention also have very good antimycotic activity. They have a very broad antimycotic activity spectrum in particular against dermatophytes and yeasts, molds and diphasic fungi (for example against Candida species such as Candida albicans, Candida glabrata) and Epidermophyton floccosum, Aspergillus species such as Aspergillus niger and Aspergillus fumigatus, Trichophyton species such as Trichophyton mentagrophytes, Microsporon species such as Microsporon canis and audouinii. The list of these fungi does by no means limit the mycotic spectrum which can be covered, but is only for illustration.

The active compounds can be used as such, in the form of their formulations or the use forms prepared therefrom, such as ready-to-use solutions, suspensions, wettable powders, pastes, soluble powders, dusts and granules. Application is carried out in a customary manner, for example by watering, spraying, atomizing, broadcasting, dusting, foaming, spreading, etc. It is furthermore possible to apply the active compounds by the ultra-low volume method, or to inject the active compound preparation or the active compound itself into the soil. It is also possible to treat the seeds of the plants.

When using the active compounds according to the invention as fungicides, the application rates can be varied within a relatively wide range, depending on the kind of application. For the treatment of parts of plants, the active compound application rates are generally between 0.1 and 10,000 g/ha, preferably between 10 and 1000 g/ha. For seed dressing, the active compound application rates are generally between 0.001 and 50 g per kilogram of seed, preferably between 0.01 and 10 g per kilogram of seed. For the treatment of the soil, the active compound application rates are generally between 0.1 and 10,000 g/ha, preferably between 1 and 5000 g/ha.

The compositions used for protecting industrial materials generally comprise the active compounds in an amount of from 1 to 95% by weight, preferably from 10 to 75% by weight.

The use concentrations of the active compounds according to the invention in the protection of materials depends on the type and the occurrence of the microorganisms to be controlled and on the composition of the material to be protected. The optimum application rate can be determined by test series. In general, the use concentrations are in the range of 0.001 to 5% by weight, preferably from 0.05 to 1.0% by weight, based on the material to be protected.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above.

Particularly preferably, plants of the plant cultivars which are in each case commercially available or in use are treated according to the invention.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products are possible which exceed the effects which were actually to be expected.

The transgenic plants or plant cultivars (i.e. those obtained by genetic engineering) which are preferably treated according to the invention include all plants which, in the genetic modification, received genetic material which imparted particularly advantageous useful properties (“traits”) to these plants. Examples of such properties are better plant growth, increased tolerance to high or low temperatures, increased tolerance to drought or to water or soil salt content, increased flowering performance, easier harvesting, accelerated maturation, higher harvest yields, better quality and/or a higher nutritional value of the harvested products, better storage stability and/or processability of the harvested products. Further and particularly emphasized examples of such properties are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, cotton, oilseed rape and also fruit plants (with the fruits apples, pears, citrus fruits and grapes), and particular emphasis is given to maize, soya beans, potatoes, cotton and oilseed rape. Traits that are emphasized are in particular increased defence of the plants against insects by toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (hereinbelow referred to as “Bt plants”). Traits that are furthermore particularly emphasized are the increased tolerance of the plants to certain herbicidally active compounds, for example imidazolinones, sulfonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucoton® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMI® (tolerance to imidazolinones) and STS® (tolerance to sulfonylureas, for example maize). Herbicide-resistant plants (plants bred in a conventional manner for herbicide tolerance) which may be mentioned also include the varieties sold under the name Clearfield® (for example maize). Of course, these statements also apply to plant cultivars having these genetic traits or genetic traits still to be developed, which plants will be developed and/or marketed in the future.

The plants listed can be treated according to the invention in a particularly advantageous manner with the compounds of the general formula (I) or the active compound mixtures according to the invention. The preferred ranges stated above for the active compounds or mixtures also apply to the treatment of these plants. Particular emphasis is given to the treatment of plants with the compounds or mixtures specifically mentioned in the present text.

Preparation and use of compounds according to the invention are illustrated by the examples below.

### PREPARATION EXAMPLES

### Example 1

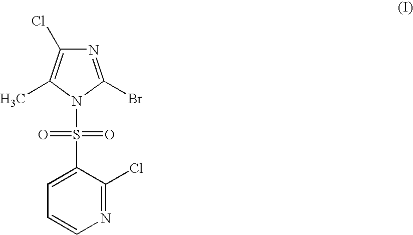

25.44 g (0.12 mol) of 2-chloro-3-pyridinesulfonyl chloride are added to a mixture of 19.55 g (0.1 mol) of 2-bromo-4-chloro-5-methyl-1H-imidazole, 120 ml acetonitril, 60 ml of dimethylformamide and 16.56 g of potassium carbonate, and the mixture is stirred at room temperature for 16 hours. 250 ml of water are added to the mixture, and the precipitate is filtered off with suction. This gives 19.8 g (53.4% of theory) of 3-[(2-bromo-4-chloro-5-methyl-1H-imidazol-1-yl)-sulfonyl]-2-chloropyridine at melting point 144-146° C.

HPLC-log P: 2.9

The azinylsulfonylimidazoles of formula (I) listed in Table 1 below are also prepared by the methods given above.

TABLE 1

| | |
|---|---|
| | (I) |
| 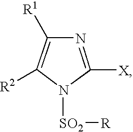 | |

| Ex. | | | | | | |
|---|---|---|---|---|---|---|
| No. | R¹ | R² | X | R | logP | m.p. (° C.) |
| 2 | —Br | —CH₃ | —Br | 2-chloro-3- | 2.97 | 154-158 |
| | | | | pyridyl | | |
| 3 | —Cl | —CH₃ | —Cl | 2-chloro-3- | 2.95 | 149-151 |
| | | | | pyridyl | | |
| 4 | —CH═CH—CH═CH— | | —CF₃ | 5-bromo-6- | 4.1 | |
| | | | | chloro-3-pyridyl | | |
| 5 | —Br | —CH₃ | —Br | 5-bromo-6- | 3.86 | |
| | | | | chloro-3-pyridyl | | |
| 6 | —Cl | —CH₃ | —Br | 2-methoxy-3- | 3.01 | |
| | | | | pyridyl | | |
| 7 | —CH₃ | —H | —CH₃ | 5-bromo-6- | 2.12 | |
| | | | | chloro-3-pyridyl | | |
| 8 | —Cl | —CH₃ | —Br | 2,6-bismethoxy- | 3.56 | |
| | | | | 3-pyridyl | | |
| 9 | —Cl | —CH₃ | —Br | 6-methoxy-3- | 3.24 | |
| | | | | pyridyl | | |
| 10 | —Cl | —CH₃ | —Cl | 2-methoxy-3- | 3.04 | |
| | | | | pyridyl | | |
| 11 | —Cl | —CH₃ | —Br | 6-chloro-3- | 3.13 | |
| | | | | pyridyl | | |
| 12 | —CH₃ | —H | —CH₃ | 6-chloro-3- | 1.41 | |
| | | | | pyridyl | | |
| 13 | —Cl | —CH₃ | —Cl | 6-chloro-3- | 3.15 | |
| | | | | pyridyl | | |
| 14 | —Cl | —CH₃ | —Br | 4,6-bismethoxy- | 2.86 | |
| | | | | 5-pyrimidyl | | |
| 15 | —Cl | —CH₃ | —Cl | 4,6-bismethoxy- | 2.9 | |
| | | | | 5-pyrimidyl | | |
| 16 | —Cl | —CH₃ | Br | 6-chloro-4- | 3.33 | |
| | | | | methyl-3-pyridyl | | |
| 17 | —Cl | —CH₃ | Cl | 6-chloro-4- | 3.39 | |
| | | | | methyl-3-pyridyl | | |
| 18 | —Cl | —CH₃ | Cl | 6-chloro-2- | 3.49 | |
| | | | | methyl-3-pyridyl | | |
| 19 | —Cl | —CH₃ | Cl | 2-chloro-6- | 3.66 | |
| | | | | methoxy-3- | | |
| | | | | pyridyl | | |
| 20 | —Cl | —CH₃ | Br | 2-chloro-6- | 3.62 | |
| | | | | methoxy-3- | | |
| | | | | pyridyl | | |
| 21 | 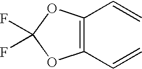 | | Cl | 2-chloro-3- pyridyl | 3.75 | |
| 22 | —Cl | —CH₃ | Cl | 2-chloro-5- | 3.31 | |
| | | | | methyl-3-pyridyl | | |
| 23 | 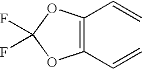 | | Cl | 6-chloro-5- bromo-5-pyridyl | 4.5 | |
| 24 | —Cl | —CH₃ | Br | 6-chloro-2- | 3.46 | |
| | | | | methyl-3-pyridyl | | |
| 25 | —Cl | —CH₃ | CN | 2-chloro-3- | 2.67 | |
| | | | | pyridyl | | |

The logP values were determined in accordance with EEC Directive 79/831 Annexe V.A8 by HPLC (gradient method, acetonitrile/0.1% aqueous phosphoric acid).

### Use Example

### Example A

Phytophthora-Test (tomato)/protective

Solvents: 24.5 parts by weight of acetone 24.5 parts by weight of dimethylacetamide

Emulsifier: 1.0 part of alkylaryl polyglycol ether

To produce a suitable preparation of active compound, one part by weight of active compound is mixed with the stated amounts of solvent and emulsifier, and the concentrate is diluted with water to the desired concentration.

To test for protective activity, young plants are sprayed with the preparation of active compound at the stated application rate. After the spray coating has dried on, the plants are inoculated with an aqueous spore suspension of Phytophthora infestans. The plants are then placed in an incubation cabin at about 20° C. and 100% relative atmospheric humidity.

Evaluation is carried out three days after the inoculation. 0% means an efficacy which corresponds to that of the control, whereas an efficacy of 100% means that no infection is observed.

Active compounds, application rates and test results are shown in the table below.

TABLE A
Phytophthora-Test (tomato)/protective

| | Application rate | |
|---|---|---|
| | of active compound | Efficacy |
| Active compound | in g/ha | in % |
| According to the invention | | |
| 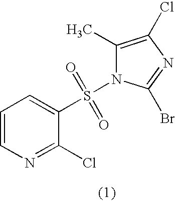 | 50 | 94 |
| 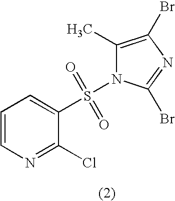 | 50 | 94 |

1. An azinylsulfonylimidazole of the formula 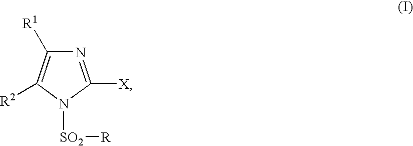
in which
R¹ represents hydrogen, halogen, cyano, nitro, alkyl, halogenoalkyl, optionally substituted aryl or optionally substituted aralkyl,
R² represents hydrogen, halogen, cyano, nitro, alkyl, halogenoalkyl, optionally substituted aryl or optionally substituted aralkyl,
R¹ and R² together with the carbon atoms to which they are attached represent an optionally substituted ring,
R represents a radical of the formula 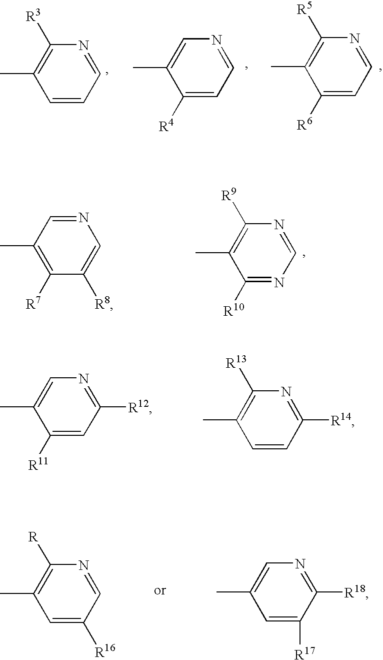
in which
R³, R⁴, R⁵, R⁶, R⁷, R⁸, R⁹, R¹⁰, R¹¹, R¹², R¹³, R¹⁴, R¹⁵, R¹⁶, R¹⁷ and R¹⁸ independently of one another represent fluorine, chlorine, bromine or methoxy,
or
R represents a radical of the formula 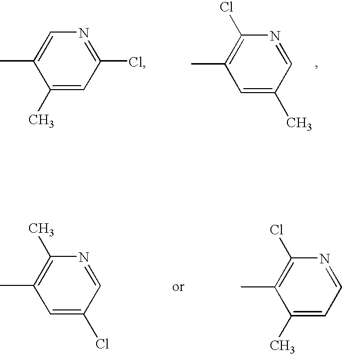
and
X represents halogen, alkyl, halogenoalkyl, cyano or thiocarbamoyl.

2. An azinylsulfonylimidazole of the formula (I) as claimed in claim 1, in which
R¹ represents hydrogen, fluorine, chlorine, bromine, iodine, cyano, nitro, alkyl having 1 to 4 carbon atoms or halogenoalkyl having 1 to 4 carbon atoms and 1 to 9 halogen atoms, or represents aryl having 6 to 10 carbon atoms, where these aryl radicals may be mono- to trisubstituted by identical or different substituents from the group consisting of fluorine, chlorine, bromine, iodine, cyano, nitro, alkyl having 1 to 4 carbon atoms, alkoxy having 1 to 4 carbon atoms and halogenoalkyl having 1 to 4 carbon atoms and 1 to 9 identical or different halogen atoms, or
represents aralkyl having 6 to 10 carbon atoms in the aryl moiety and 1 to 4 carbon atoms in the alkyl moiety, where these radicals may be mono- to trisubstituted in the aryl moiety by identical or different substituents from the group consisting of fluorine, chlorine, bromine, iodine, cyano, nitro, alkyl having 1 to 4 carbon atoms, alkoxy having 1 to 4 carbon atoms and/or halogenoalkyl having 1 to 4 carbon atoms and 1 to 9 identical or different halogen atoms,
R² represents hydrogen, fluorine, chlorine, bromine, iodine, cyano, nitro, alkyl having 1 to 4 carbon atoms, or halogenoalkyl having 1 to 4 carbon atoms and 1 to 9 halogen atoms, or represents aryl having 6 to 10 carbon atoms, where the aryl radical may be mono- to trisubstituted by identical or different substituents from the group consisting of fluorine, chlorine, bromine, iodine, cyano, nitro, alkyl having 1 to 4 carbon atoms, alkoxy having 1 to 4 carbon atoms and halogenoalkyl having 1 to 4 carbon atoms and 1 to 9 identical or different halogen atoms, or represents aralkyl having 6 to 10 carbon atoms in the aryl moiety and 1 to 4 carbon atoms in the alkyl moiety, where these radicals may be mono- to trisubstituted in the aryl moiety by identical or different substituents from the group consisting of fluorine, chlorine, bromine, iodine, cyano, nitro, alkyl having 1 to 4 carbon atoms, alkoxy having 1 to 4 carbon atoms and halogenoalkyl having 1 to 4 carbon atoms and 1 to 9 identical or different halogen atoms, or
R¹ and R² together with the carbon atoms to which they are attached represent a five- or six-membered ring which may be mono- to trisubstituted by identical or different substituents from the group consisting of halogen and alkyl having 1 to 4 carbon atoms, or
R¹ and R² together with the carbon atoms to which they are attached represent a bicyclic ring system having 5 or 6 members per ring, where the bicyclic ring system may be mono- to tetrasubstituted by identical or different substituents from the group consisting of halogen and alkyl having 1 to 4 carbon atoms,
R represents a radical of the formula 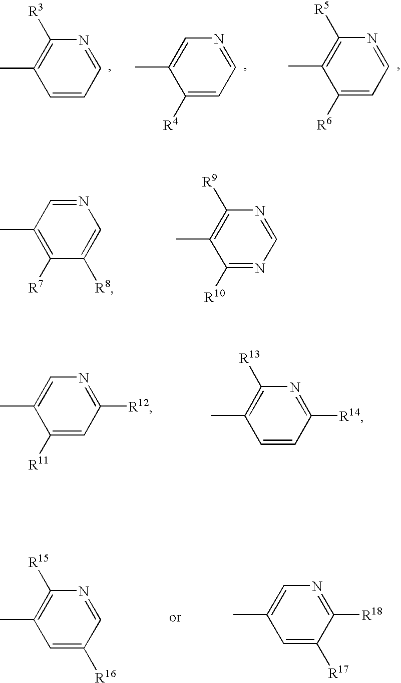
in which
R³, R⁴, R⁵, R⁶, R⁷, R⁸, R⁹, R¹⁰, R¹¹, R¹², R¹³, R¹⁴, R¹⁵, R¹⁶, R¹⁷ and R¹⁸ independently of one another represent fluorine, chlorine, bromine or methoxy,
or
R represents a radical of the formula 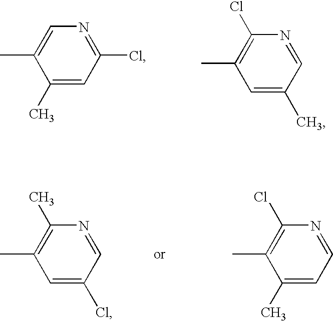
and
X represents fluorine, chlorine, bromine, alkyl having 1 to 4 carbon atoms, halogenoalkyl having 1 to 4 carbon atoms and 1 to 9 halogen atoms, cyano or thiocarbamoyl.

3. An azinylsulfonylimidazole of the formula (I) as claimed in claim 1, in which
R¹ represents hydrogen, fluorine, chlorine, bromine, cyano, nitro, methyl, trifluoromethyl, ethyl, phenyl, tolyl, methoxyphenyl, chlorophenyl, trifluoromethylphenyl, benzyl, methylbenzyl, methoxybenzyl, chlorobenzyl or trifluoromethylbenzyl,
R² represents hydrogen, fluorine, chlorine, bromine, cyano, nitro, methyl, trifluoromethyl, ethyl, phenyl, tolyl, methoxyphenyl, chlorophenyl, trifluoromethylphenyl, benzyl, methylbenzyl, methoxybenzyl, chlorobenzyl or trifluoromethylbenzyl, or
R¹ and R² together with the carbon atoms to which they are attached represent phenylene which may be mono- to trisubstituted by identical or different substituents from the group consisting of fluorine, chlorine, bromine, methyl, ethyl and trifluoromethyl, or
R¹ and R² together with the carbon atoms to which they are attached represent methylenedioxyphenylene which may be mono- or disubstituted at the methylene group by identical or different substituents from the group consisting of fluorine, chlorine, bromine, methyl and trifluoromethyl, or
R¹ and R² together with the carbon atoms to which they are attached represent ethylenedioxyphenylene which may be mono- to tetrasubstituted by identical or different substituents from the group consisting of fluorine, chlorine, bromine, methyl and trifluoromethyl,
R represents a radical of the formula 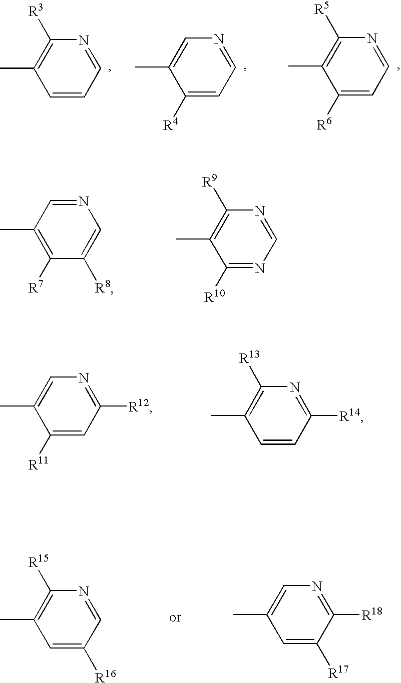
in which
R³, R⁴, R⁵, R⁶, R⁷, R⁸, R⁹, R¹⁰, R¹¹, R¹², R¹³, R¹⁴, R¹⁵, R¹⁶, R¹⁷ and R¹⁸ independently of one another represent fluorine, chlorine, bromine or methoxy,
or
R represents a radical of the formula 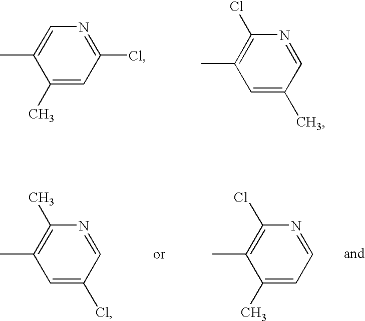
X represents fluorine, chlorine, bromine, methyl, trifluoromethyl, cyano or thiocarbamoyl.

4. An azinylsulfonylimidazole as claimed in claim 1, wherein said azinylsulfonylimidazole is represented by the formula

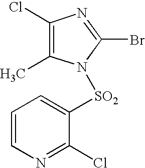

5. An azinylsulfonylimidazole as claimed in claim 1, wherein said azinylsulfonylimidazole is represented by the formula

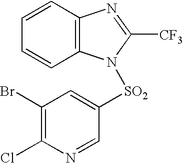

6. An azinylsulfonylimidazole as claimed in claim 1, wherein said azinylsulfonylimidazole is represented by the formula

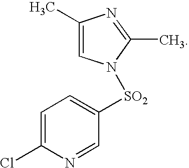

7. An azinylsulfonylimidazole as claimed in claim 1, wherein said azinylsulfonylimidazole is represented by the formula

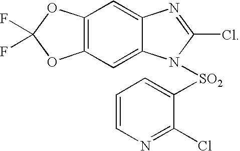

8. A process for preparing an azinylsulfonylimidazole of the formula (I) as claimed in claim 1, comprising reacting an imidazole of the formula 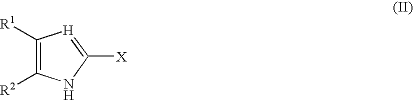
in which
R¹, R² and X are as defined in claim 1,
with a sulfonyl halide of the formula
R—SO₂—X¹ (III)
in which
R is as defined in claim 1 and
X¹ represents chlorine or bromine,
optionally in the presence of an acid binder and optionally in the presence of a diluent.

9. A composition for controlling unwanted microorganisms, comprising at least one azinylsulfonylimidazole of the formula (I) as claimed in claim 1 and one or more extenders and/or surfactants.

10. (Cancelled)

11. A method for controlling unwanted microorganisms, comprising applying an azinylsulfonylimidazole of the formula (I) as claimed in claim 1 to the microorganisms and/or their habitats.

12. A process for preparing a composition for controlling unwanted microorganisms comprising mixing an azinylsulfonylimidazole of the formula (I) as claimed in claim 1 with one or more extenders and/or surfactants.
